(19) United States
(12) Patent Application Publication
(10) Pub. No.: US 2005/0032656 A1
(43) Pub. Date: Feb. 10, 2005

(54) Use of alkyl glucosides to obtain or enhance selectivity of cleaning formulations

(75) Inventors: Manfred Strassner, Hamburg (DE); Stephan Ruppert, Hamburg (DE); Andreas Schepky, Hamburg (DE); Jens-Peter Vietzke, Hamburg (DE); Harald Albrecht, Hamburg (DE); Urte Koop, Hamburg (DE); Ralph Schimpf, Bonningstedt (DE); Joachim Ennen, Hamburg (DE); Claudius Rapp, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/840,891

(22) Filed: May 7, 2004

Related U.S. Application Data
(63) Continuation of application No. PCT/EP02/10818, filed on Sep. 26, 2002.

(30) Foreign Application Priority Data
Nov. 7, 2001 (DE) 101 54 628.9

(51) Int. Cl. A61K 7/50

(52) U.S. Cl. 510/130; 510/470

(57) ABSTRACT

The invention is a cosmetic or dermatological cleaning preparation, comprising one or more interface-active substances selected from the group consisting of alkyl glucosides having the formula 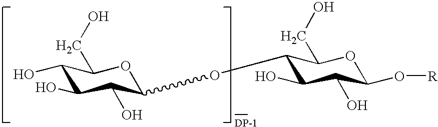 where R is a branched or unbranched alkyl radical having 4 to 24 carbon atoms and where \overline{DP} is an average degree of glucosylation of up to 2. The preparation can be applied to the skin to improve a skin condition. In particular, the alkyl glucosides of the invention can be used in cosmetic or dermatological preparations to obtain or enhance selectivity the cosmetic or dermatological cleaning formulations.

### CROSS-REFERENCE TO RELATED APPLICATIONS

This is a continuation application of PCT/EP02/10818, filed Sep. 26, 2002, which is incorporated herein by reference in its entirety, and also claims the benefit of German Priority Application No. 101 54 628.9, filed Nov. 7, 2001.

### FIELD OF THE INVENTION

The present invention relates to the use of alkyl glucosides to obtain or enhance the selectivity of cosmetic or dermatological cleaning preparations.

### BACKGROUND OF THE INVENTION

Even cleansing of the skin using a simple water bath—without the addition surfactants—initially causes the horny layer of the skin to swell. The degree of this swelling depends inter alia on the bathing time and temperature. At the same time, water-soluble substances are washed off or out, such as, for example, water-soluble constituents of dirt, but also substances endogenous to the skin which are responsible for the water-binding capacity of the horny layer. In addition, as a result of surface-active substances which are endogenous to the skin, fats in the skin (surface and barrier lipids) are also dissolved and washed out to a certain degree.

While the combined removal of surface dirt and surface lipids which primarily originate from the sebaceous glands and are decisively responsible for the appearance of greasy skin is entirely desirable in cosmetic terms, the washing-out of barrier lipids can impair the function of the skin barrier, which is associated with a loss of moisture from the skin. This is accompanied by a sporadic increase in the transepidermal water loss (TEWL) and a sporadic reduction in skin moisture. Primarily for products for the cleaning of sensitive facial skin and here particularly in the case of products for greasy skin, as selective as possible a removal of surface dirt, make-up and skin sebum is particularly important for the consumer. On the other hand, the facial skin in particular requires particularly gentle cleaning.

In healthy skin, the disturbances caused by washing are generally limited in terms of time since the protective mechanisms of the skin are able to readily compensate for such slight disturbances to the upper layers of the skin. However, even in the case of nonpathological deviations from the norm, e.g. as a result of wear damage or irritations caused by the environment, photodamage, aging skin etc., the protective mechanism of the surface of the skin is impaired. In some instances, it is then no longer able by itself to fulfill its function and has to be regenerated by external measures. There has therefore been no lack of attempts to find suitable cleaning preparations for better and more rapid regeneration of the skin which help the skin to retain its natural balance.

Known compositions for the cleaning and simultaneous care of the skin are, for example, oil-containing cleaning preparations or cleaning preparations comprising various refatting agents or skin moisturizers, which are intended to refat or remoisturize the skin at the same time as cleaning it. For this purpose, the prior art recognizes, for example, oil bath preparations of various types and also shower oils, cleansing creams and the like.

The main disadvantage of such preparations is that some of the barrier lipids are initially removed and the skin is then refatted with the help of the added oil components. In addition, such cleaning preparations are washed off following application, meaning that only small amounts of the additives used remain on the skin.

### SUMMARY OF THE INVENTION

It was therefore an object of the present invention to provide cosmetic or dermatological preparations which significantly improve the condition of the skin, in particular reduce the roughness of the skin and which are characterized by a selectivity of the cleaning performance.

It was surprising and could in no way have been foreseen by the person skilled in the art that the use of one or more interface-active substances chosen from the group of alkyl glucosides which are characterized by the structural formula 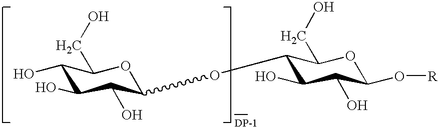
where R is a branched or unbranched alkyl radical having 4 to 24 carbon atoms and where \overline{DP} is an average degree of glucosylation of up to 2, to obtain or enhance the selectivity of cosmetic or dermatological cleaning preparations would overcome the disadvantages of the prior art.

### DETAILED DESCRIPTION OF THE PREFERRED EMBODIMENTS

Selective cleaning preparations within the meaning of the present invention wash significantly more surface or sebum lipids out than barrier lipids. Thus, if the ratio of washed out surface or sebum lipids to washed out barrier lipids is defined, then this is significantly greater than 1 for preparations comprising alkyl glucosides used according to the invention. The increase in the selectivity of a cleaning preparation can be determined according to the invention, for example, relative to water. According to the present invention, it is possible to formulate products which remove dirt and excess sebum with high selectivity and in so doing conserve the lipids endogenous to the skin which are essential for preventing the skin from drying out.

The value \overline{DP} represents the degree of glucosylation of the alkyl glucosides used according to the invention and is defined as

$$\overline{DP} = \frac{{p}_{1}}{100} A1 + \frac{{p}_{2}}{100} A2 + \frac{{p}_{3}}{100} A3 + … = {\sum }_{i}^{} \frac{{p}_{i}}{100} Ai$$

Here, p₁, p₂, p₃ . . . and pi represent the fraction of mono-, di-, tri- . . . i-times glucosylated products in percentages by weight. According to the invention, products with degrees of glucosylation of 1-2, particularly advantageously from 1.1 to 1.5, very particularly advantageously of about 1.3 are advantageously chosen.

The value DP takes into account the fact that alkyl glucosides generally represent mixtures of monoglucosides and oligoglucosides as a result of the preparation. According to the invention, a relatively high content of monoglucosides, typically in the order of magnitude of from 40 to 70% by weight, based on the total weight of the mixture, is advantageous.

R is advantageously chosen from the group of unbranched alkyl radicals, preference being given to the myristyl radical, the cetyl radical, the stearyl radical and the eicosyl radical. Particular preference is given to lauryl glucoside, decyl glycoside and cocoglycoside.

Alkyl glucosides (also: alkyl polyglycosides) used according to the invention are obtainable by processes as are described, for example, in DE-A 40 40 655 and other specifications. They are available commercially from various manufacturers.

For example, it is advantageous to use mixtures of stearyl glucoside and cetyl glucoside. Such mixtures are available commercially, for example, under the trade name Tego® Care SG from Th. Goldschmidt KG.

The total amount of one or more interface-active glucose derivatives used according to the invention in the finished cosmetic or dermatological preparations is advantageously chosen from the range from 0.1 to 25.0% by weight, preferably 0.1 to 15.0% by weight, in each case based on the total weight of the preparations.

The cleaning compositions comprising alkyl glucosides used according to the invention can have the customary composition and serve for the cosmetic and/or dermatological cleaning of the skin and/or the hair and as make-up product for decorative cosmetics.

For use, the compositions comprising alkyl glucosides used according to the invention are applied to the skin and/or the hair in a sufficient amount in the manner customary for cosmetics.

The cleaning preparations for the purposes of the present invention advantageously comprise one or more washing-active surfactants in the following four groups A to D:

A. Anionic Surfactants

Anionic surfactants to be used advantageously are:

acylamino acids (and salts thereof), such as

1. acyl glutamates, for example sodium acyl glutamate, di-TEA-palmitoyl aspartate and sodium caprylic/capric glutamate,
2. acylpeptides, for example palmitoyl-hydrolyzed milk protein, sodium cocoyl-hydrolyzed soya protein and sodium/potassium cocoyl-hydrolyzed collagen,
3. sarcosinates, for example myristoyl sarcosine, TEA-lauroyl sarcosinate, sodium lauroyl sarcosinate and sodium cocoyl sarcosinate,
4. taurates, for example sodium lauroyl taurate and sodium methylcocoyl taurate,
   carboxylic acids and derivatives, such as
1. carboxylic acids, for example lauric acid, aluminum stearate, magnesium alkanolate and zinc undecylenate,
2. ester carboxylic acids, for example calcium stearoyl lactylate, laureth-6 citrate and sodium PEG-4 lauramide carboxylate,
3. ethercarboxylic acids, for example sodium laureth-13 carboxylate and sodium PEG-6 cocamide carboxylate,
   phosphoric esters and salts, such as, for example, DEA-oleth-10 phosphate and dilaureth-4 phosphate,
   sulfonic acids and salts, such as
1. acyl isethionates, e.g. sodium/ammonium cocoyl isethionate,
2. alkylarylsulfonates,
3. alkylsulfonates, for example sodium cocomonoglyceride sulfate, sodium C₁₂₋₂₄ olefinsulfonate, sodium lauryl sulfoacetate and magnesium PEG-3 cocamide sulfate,
4. sulfosuccinates, for example dioctyl sodium sulfosuccinate, disodium laureth sulfosuccinate, disodium lauryl sulfosuccinate and disodium undecyleneamido-MEA sulfosuccinate and
   sulfuric esters, such as
1. alkyl ether sulfate, for example sodium, ammonium, magnesium, MIPA, TIPA laureth sulfate, sodium myreth sulfate and sodium C₁₂₋₁₃ pareth sulfate,
2. alkyl sulfates, for example sodium, ammonium and TEA lauryl sulfate.
   B. Cationic Surfactants
   Cationic surfactants which can be used advantageously are
1. alkylamines,
2. alkylamidazoles,
3. ethoxylated amines and
4. quaternary surfactants.

Quaternary surfactants contain at least one N atom which is covalently bonded to 4 alkyl or aryl groups. Irrespective of the pH, this leads to a positive charge. Benzalkonium chloride, alkylbetaine, alkylamidopropylbetaine and alkylamidopropyl-hydroxysultaine are advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example aminopropylalkylglutamide, alkylaminopropionic acid, sodium alkylamidodipropionate and lauroamphocarboxyglycinate.
3. N-alkyl- or N-alkenylbetaines with at least 12 carbon atoms, such as, for example, laurylamidopropylbetaine and oleylamidopropylbetaine.
   D. Nonionic Surfactants
   Nonionic surfactants which can be used advantageously are
1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coco glycoside.
6. Sucrose esters, sucrose ethers
7. Polyglycerol esters, diglycerol esters, monoglycerol esters
8. Methylglucose esters, esters of hydroxy acids.

The cleaning preparations for the purposes of the present invention particularly advantageously comprise one or more washing-active surfactants according to the invention from the group of surfactants which have an HLB value of more than 25, very particularly those which have an HLB value of more than 35.

It is advantageous for the purposes of the present invention when the content of one or more washing-active surfactants in the cosmetic or dermatological cleaning preparation is chosen from the range from 2.0 to 25% by weight, very particularly advantageously from 2.5 to 15% by weight, in each case based on the total weight of the preparation.

The compositions for the purposes of the present invention further advantageously comprise water and optionally the additives customary in cosmetics, e.g. preservatives, preserving aids, bactericides, perfumes, dyes, pigments which have a coloring effect, moisturizing and/or humectant substances, fillers which improve the feel on the skin, fats, oils, waxes or other customary constituents of a cosmetic or dermatological formulation.

Advantageous preservatives for the purpose of the present invention are, for example, formaldehyde donors (such as, for example, DMDM hydantoin, which is available, for example, under the trade name Glydant™ from Lonza), iodopropyl butylcarbamates (e.g. those available under the trade names Glycacil-L, Glycacil-S from Lonza and/or Dekaben LMB from Jan Dekker), parabens (i.e. p-hydroxybenzoic alkyl esters, such as methyl-, ethyl-, propyl- and/or butylparaben), phenoxyethanol, ethanol, benzoic acid and the like. According to the invention, the preservative system usually also advantageously comprises preserving aids, such as, for example, octoxyglycerol, glycine soya, etc.

The water phase of the preparations for the purposes of the present invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydroxyacetone, and in particular one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination. Moisturizers can also preferably be used.

Moisturizers is the term used to described substances or mixtures of substances which, following application or distribution on the surface of the skin, confer on cosmetic or dermatological preparations the property of reducing the moisture loss by the horny layer (also called transepidermal water loss (TEWL) and/or have a beneficial effect on the hydration of the horny layer.

Advantageous moisturizers for the purposes of the present invention are, for example, glycerol, lactic acid and/or lactates, in particular sodium lactate, butylene glycol, propylene glycol, biosaccharide gum-1, glycine soya, ethylhexyloxyglycerol, pyrrolidonecarboxylic acid and urea. In addition, is particularly advantageous to use polymeric moisturizers from the group of polysaccharides which are soluble in water and/or swellable in water and/or gellable using water. Particularly advantageous are, for example, hyaluronic acid, chitosan and/or a fucose-rich polysaccharide, which is listed in Chemical Abstracts under the registry number 178463-23-5 and is available, for example, under the name Fucogel® 1000 from SOLABIA S.A.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic and/or dermatological applications.

For the purposes of the present invention, water-soluble antioxidants may be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin and/or the hair against oxidative stress.

Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatine, taurine and/or β-alanine.

The examples below are intended to illustrate the present invention. The numerical values in the examples are percentages by weight, based on the total weight of the respective preparations.

### EXAMPLES

### Formulation Examples

| | 1 | 2 | 3 | 4 | 5 |
|---|---|---|---|---|---|
| Decyl glucoside | 0.25 | — | 0.25 | 7.5 | 15 |
| Lauryl glucoside | — | 0.25 | 0.25 | — | — |
| Sodium lauryl ether | 1.5 | — | 1.5 | — | — |
| sulfate | | | | | |
| Sodium myristyl ether | — | 1.5 | — | — | — |
| sulfate | | | | | |
| Sodium methyl cocoyl | 0.6 | 0.5 | 0.3 | — | — |
| taurate | | | | | |
| Glycerol | 2.0 | 2.0 | 1.0 | — | — |
| Propylene glycol | — | — | — | 18 | 18 |
| Butylene glycol | — | — | — | 5 | 5 |
| Sodium carbomer | 1.2 | 1.2 | 1.2 | — | — |
| Sodium acrylate/C10-30 | — | — | — | 1.3 | 1.3 |
| alkyl acrylate | | | | | |
| crosspolymer | | | | | |
| Xanthan gum | 0.25 | 0.25 | 0.2 | — | — |
| PEG-40 hydrogenated | — | — | — | 1 | 1 |
| castor oil | | | | | |
| PEG-7 glyceryl cocoate | 0.5 | — | 0.5 | — | — |
| Parabens | 0.2 | 0.2 | 0.15 | 0.15 | 0.15 |
| EDTA | — | — | — | 0.2 | 0.2 |
| Benzophenone-4 | 0.05 | 0.05 | 0.04 | 0.03 | 0.03 |
| Methyldibromo- | 0.05 | 0.05 | 0.04 | — | — |
| glutaronitrile | | | | | |
| Phenoxyethanol | 0.6 | 0.6 | 0.5 | 0.35 | 0.35 |
| Perfume | qs | qs | qs | qs | qs |
| Water | ad 100 | ad 100 | ad 100 | ad 100 | ad 100 |

1. A cosmetic or dermatological cleaning preparation, comprising one or more interface-active substances selected from the group consisting of alkyl glucosides having the formula 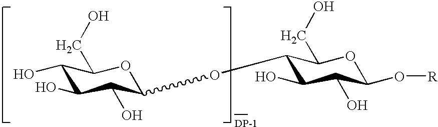
where R is a branched or unbranched alkyl radical having 4 to 24 carbon atoms and where \overline{DP} is an average degree of glucosylation of up to 2.

2. The preparation as claimed in claim 1, wherein the content of the one or more alkyl glucosides is 0.1 to 15% by weight, based on the total weight of the preparation.

3. The preparation as claimed in claim 1, wherein the content of the one or more alkyl glucosides is 0.25 to 10% by weight, based on the total weight of the preparation.

4. The preparation as claimed in claim 1, further comprising one or more surfactants, wherein the total concentration of the one or more surfactants is 1.5 to 20% by weight, based on the total weight of the preparation.

5. The preparation as claimed in claim 1, further comprising one or more surfactants, wherein the total concentration of the one or more surfactants is 2.0 to 15% by weight, based on the total weight of the preparation.

6. The preparation as claimed in claim 1, wherein the alkyl glucosides are selected from the group consisting of decyl glucosides and lauryl glucosides.

7. The preparation as claimed in claim 1, wherein the alkyl glucosides are selected from the group consisting of mixtures of decyl glucosides and lauryl glucosides.

8. The preparation as claimed in claim 1, further comprising one or more cosurfactants selected from the group consisting of alkyl ether sulfates and alkyltaurates.

9. The preparation as claimed in claim 1, further comprising one or more gel formers selected from the group consisting of polyacrylates, acrylate copolymers and polysaccharides.

10. The preparation as claimed in claim 1, said preparation being suitable for foaming using a pump foamer.

11. A method for improving a condition of the skin, the method comprising applying to the skin, a cosmetic or dermatological cleaning preparation comprising one or more interface-active substances selected from the group consisting of alkyl glucosides having the formula 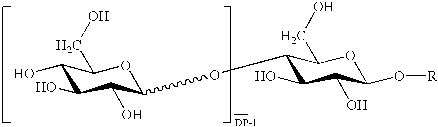
where R is a branched or unbranched alkyl radical having 4 to 24 carbon atoms and where \overline{DP} is an average degree of glucosylation of up to 2.

12. The method as claimed in claim 11, wherein said preparation is applied to the skin to reduce the roughness of the skin.

13. The method as claimed in claim 11, wherein the content of the one or more alkyl glucosides in the preparation is 0.1 to 15% by weight, based on the total weight of the preparation.

14. The method as claimed in claim 11, wherein the content of the one or more alkyl glucosides in the preparation is 0.25 to 10% by weight, based on the total weight of the preparation.

15. The method as claimed in claim 11, wherein the alkyl glucosides are selected from the group consisting of decyl glucosides and lauryl glucosides.

16. The method as claimed in claim 11, wherein the alkyl glucosides are selected from the group consisting of mixtures of decyl glucosides and lauryl glucosides.

17. The method as claimed in claim 11, wherein said applying step comprises applying the preparation by foaming the preparation using a pump foamer.

18. The method as claimed in claim 11, wherein said applying step comprises applying the preparation to the body for cleaning the body.

19. The method as claimed in claim 11, wherein said applying step comprises applying the preparation to the face for cleaning the face.

20. The method as claimed in claim 11, wherein said applying step comprises applying the preparation to the scalp for cleaning the scalp.

21. A method for obtaining or enhancing the selectivity of a cosmetic or dermatological cleaning preparation, the method comprising adding to the cosmetic or dermatological cleaning preparation comprising one or more interface-active substances selected from the group consisting of alkyl glucosides having the formula 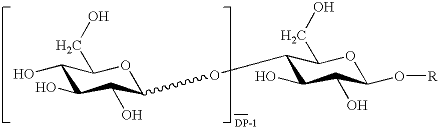
where R is a branched or unbranched alkyl radical having 4 to 24 carbon atoms and where \overline{DP} is an average degree of glucosylation of up to 2.

22. The method according to claim 21, wherein the selectivity of the preparation is enhanced such that when the preparation is applied to the skin that the preparation washes out significantly more surface or sebum lipids than barrier lipids.

23. The method according to claim 21, wherein the selectivity of the preparation is enhanced such that when the preparation is applied to the skin that the ratio of washed out surface or sebum lipids to washed out barrier lipids is greater than 1.

24. The method according to claim 21, wherein the selectivity of the preparation is enhanced such that when the preparation is applied to the skin that the ratio of washed out surface or sebum lipids to washed out barrier lipids is significantly greater than 1.

25. The method as claimed in claim 21, wherein the content of the one or more alkyl glucosides in the preparation is 0.1 to 15% by weight, based on the total weight of the preparation.

26. The method as claimed in claim 21, wherein the content of the one or more alkyl glucosides in the preparation is 0.25 to 10% by weight, based on the total weight of the preparation.

27. The method as claimed in claim 21, wherein the alkyl glucosides are selected from the group consisting of decyl glucosides and lauryl glucosides.

28. The method as claimed in claim 21, wherein the alkyl glucosides are selected from the group consisting of mixtures of decyl glucosides and lauryl glucosides.
